(19) United States
(12) Patent Application Publication
(10) Pub. No.: US 2020/0024674 A1
(43) Pub. Date: Jan. 23, 2020

(54) BIO-DEGRADABLE COMPOSITION AND/OR PROBIOTIC BIOCHEMICALS AND METHODS OF USE IN LEATHER TANNING PROCESSES

(71) Applicant: Sustainable Community Development, L.L.C., D/B/A SCD Probiotics, Kansas City, MO (US)

(72) Inventors: Matthew T. Wood, Kansas City, MO (US); Narin Tipsrisukond, Kansas City, MO (US); Juan Carlos Castell, Barcelona (ES)

(21) Appl. No.: 16/477,387

(22) PCT Filed: Jan. 12, 2018
(86) PCT No.: PCT/US18/13618

§ 371 Date: Jul. 11, 2019

Related U.S. Application Data
(60) Provisional application No. 62/446,195, filed on Jan. 13, 2017.

(51) Int. Cl. C14C 1/06; C12N 1/20; C12P 1/04; C12P 39/00; C14C 9/00; C14C 3/26; C14C 1/04; C14C 1/08

(52) U.S. Cl. CPC C14C 1/065; C12N 1/20; C12P 1/04; C14C 1/08; C14C 9/00; C14C 3/26; C14C 1/04; C12P 39/00

(57) ABSTRACT

Use of probiotic technology to support tanners to reduce waste and increase the value of by-products reducing the need of synthetic chemicals, improving the quality of effluents and reducing the carbon footprint to the environment. Probiotics or beneficial microorganisms are used to obtain biochemicals through a controlled fermentation of natural ingredients. The result of the process is a consortium of metabolites including viable probiotic microorganisms with properties very similar to traditional leather auxiliaries extensively needed during leather production, eliminating or reducing the need for use of toxic chemicals and compositions.

### CROSS-REFERENCE TO RELATED APPLICATIONS

The present application claims the priority benefit of U.S. Provisional Patent Application Ser. No. 62/446,195, filed Jan. 13, 2017, entitled BIO-DEGRADABLE BIOCIDE COMPOSITION AND/OR PROBIOTIC ADJUVANT METHOD OF USE IN LEATHER TANNING PROCESSES, incorporated by reference in its entirety herein.

### BACKGROUND OF THE INVENTION

### Field of the Invention

The present invention relates to microorganism compositions and methods of using. In particular, the present invention relates to a microorganism consortia composition including lactic acid, sulfide-utilizing, probiotic, and phototrophic microorganisms co-cultured to produce a composition useful in the hide tanning industry as a biodegradable composition, and/or probiotic biochemicals at various stages of leather tanning including raw hide preservation, pre-soaking, soaking, liming, de-liming, bating, degreasing, tanning, wetting back, retanning, fat liquoring. These compositions eliminate or reduce the need for use of toxic chemicals such as Bactericides, Fungicides, Sodium sulfide, Chromium, Synthetic fats, Enzymes, Chemical surfactants, and the like.

### Description of Related Art

Environmental awareness, resource constraints, and general public opinion are increasing the demand for efficient green technologies and products. Such green technologies and products are those that promote sustainability and have minimal impact on the environment. One area that is being exploited to develop green technology and products is the use of microorganisms and their specialized properties. Microorganisms have been used in agriculture, animal health, human health, and waste management. In agriculture, microorganisms are used to enhance composting and soil amendment. In animals and humans, beneficial bacteria, known as probiotics, are used to prevent illness caused by harmful bacteria invading the natural flora. In waste management, microorganisms are used to accelerate waste decomposition and degrade odorous compounds.

While the use of microorganisms is being exploited, such use is hindered by stability, storage, and efficiency issues. Accordingly, there is a need to develop microorganism-based technologies and products that are stable under various conditions, have an appreciable shelf life, and can be easily used.

### SUMMARY OF THE INVENTION

Described herein are probiotic biochemical composition(s) comprising a microorganism fermentation broth with a defined fingerprinting profile from metagenomics and metabolomics analysis. The probiotic biochemical composition(s) comprise Total Lactic Acid Bacteria counts of between about 1.0E+4 to about 1.0E+6 CFU/mL, Purple sulfur bacteria, and are essentially free of pathogenic or putrefactive microorganisms. The fermentation broth is obtained by fermentation of probiotic microorganisms in a medium under defined fermentation conditions. The probiotic biochemical composition (final product) has a pH value of <4.0 with activity in pH range from 2 to 12, percent total acidity between about 0.6 and about 3.2, and an Emulsification Index value between about 40% and about 75%.

Methods of producing leather are also described herein. The methods generally comprise contacting an animal hide with a probiotic biochemical composition according to the various embodiments of the invention.

Embodiments of the invention are also concerned with methods of reducing sodium sulfide in the production of leather products. The methods generally comprise unhairing an animal hide by contacting the hide with lime, a probiotic biochemical composition, and an amount of sodium sulfide for an effective period of time to release hair from the hide, wherein the amount of sodium sulfide used in unhairing is reduced by at least 30%. The probiotic biochemical composition comprises Total Lactic Acid Bacteria counts of between about 1.0E+4 to about 1.0E+6 CFU/mL, Purple sulfur bacteria, and are essentially free of pathogenic or putrefactive microorganisms. The fermentation broth is obtained by fermentation of probiotic microorganisms in a medium under defined fermentation conditions. The probiotic biochemical composition (final product) has a pH value of <4.0 with activity in pH range from 2 to 12, percent total acidity between about 0.6 and about 3.2, and an Emulsification Index value between about 40% and about 75%.

Use of a probiotic biochemical composition in the production of leather products is also described herein, wherein the probiotic biochemical composition comprises Total Lactic Acid Bacteria counts of between about 1.0E+4 to about 1.0E+6 CFU/mL, Purple sulfur bacteria, and is essentially free of pathogenic or putrefactive microorganisms. The fermentation broth is obtained by fermentation of probiotic microorganisms in a medium under defined fermentation conditions. The probiotic biochemical composition (final product) has a pH value of <4.0 with activity in pH range from 2 to 12, percent total acidity between about 0.6 and about 3.2, and an Emulsification Index value between about 40% and about 75%.

The present invention describes the use of probiotic technology to support tanners to reduce waste and increase the value of by-products reducing the need of synthetic chemicals, improving the quality of effluents and reducing impact on the environment. Probiotics or beneficial microorganisms are used to obtain biochemicals through a controlled fermentation of natural ingredients. The result of the process is a consortium of metabolites with viable probiotic microorganisms and properties very similar to traditional leather auxiliaries extensively used during the leather making process.

Metabolites produced by the probiotic consortia are characterized by strong polar groups, mainly anionic, and active functional radicals that enable them to be combined with other organic molecules.

The properties of the metabolites for industrial applications are provided by the consortia of biochemicals present in the preparation, including, without limitation: high reactivity to combine other molecules through their strong and multiple functional radicals, with powerful properties even at very low concentration; small molecules that can be disseminated to the tiny spaces existing between collagen fibrils of leather; bactericidal-like properties due to the presence of carboxylic acids; and hydrotropic properties that provides hydration and increase solubility of the substances to which they combine.

It is an object of the invention to provide a biodegradable composition and/or probiotic biochemicals of a microorganism consortium, where the fermentation product(s) of the microorganism consortium are mixed with suitable proportions of a deodorizer extracted from essential oils and additives to keep the entire formulation stable over a period of one year or more.

In one aspect, the microorganism consortium is initially fermented to produce a fermentation product. The composition may be fermented for about 1, 2, 3, 4, 5, 6, 7, 8, 9, 10, 11, 12, 13, 14, 15, 16, 17, 18, 19, 20, 21, 22, 23, 24, 25, or more days. Preferably, the composition is fermented for at least about 15 to about 23 days. More preferably, the composition is fermented for at least 21 days. In some embodiments, the fermentation product is combined with additional ingredients, such as deodorizers, stabilizers, additives, etc. to yield the final product.

The composition is characterized by a fermentation broth of a microorganism consortium, where the microorganism consortium comprises probiotic microorganisms, and more specifically at least 3 different microorganisms. In one or more embodiments, at least one microorganism is a sulfide-utilizing microorganism. Additional microorganisms include one or more of lactic acid, probiotic, and/or phototrophic microorganisms, as well as mixtures thereof. The microorganisms may include species of bacteria, yeast, fungi, mold, or mixtures thereof.

In one or more embodiments, the composition includes at least one species of microorganism from lactic acid, probiotic, phototrophic, and purple non-sulfur bacteria species.

The fermentation broth is obtained by fermentation of selected microorganisms in a selected medium containing a source of carbon (e.g., fruit juices, unrefined cane sugar, beet sugar, molasses, and/or natural sugar sources), under defined fermentation conditions. Exemplary microorganism starting consortiums and fermentation processes are described in detail in U.S. Pat. No. 9,096,836, filed Mar. 14, 2013, incorporated by reference herein in its entirety.

In one aspect, the resulting probiotic composition (with or without additives) includes a total number of microorganisms of about 1 to about 1 million colony forming units (CFU) per milliliter. Preferably, the composition includes a total number of microorganisms of about 100,000 to about 800,000 CFU per milliliter. More preferably, the composition includes a total number of microorganisms of about 250,000 to about 600,000 CFU per milliliter. Most preferably, the composition includes a total number of microorganisms of about 300,000 CFU per milliliter.

In one aspect, the composition includes living microorganisms or non-living microorganisms. In another aspect, the composition includes both living and non-living microorganisms. Compositions containing non-living microorganisms may also contain extracts of the microorganisms. Such extracts may be considered a liquid fermentation product of the living microorganisms. The extracts of microorganisms include, by way of example, organic acids such as acetic acid, lactic acid, and/or fatty acids, as well as small chain peptides, biopolymers and other compounds such as vitamins and polyols that are produced by microorganisms. The extracts are capable of eliciting an effect on an environment regardless of the living status of the microorganism in the final composition.

In yet another aspect, the present invention provides compositions mixed in suitable proportions with a deodorizer extracted from essential oils (e.g., lavender oil, lemon oil, lemon grass oil, and the like, or combinations thereof). In another aspect, the composition comprises additives, such as stabilizers (e.g., nonionic surfactants, emulsifiers, and the like) to increase stability of the formulation over a period of one year or more.

It is yet another object of the invention to have a metagemonics profile characterized by the relative amount (i.e., Relative Richness) of microorganisms in the composition, such microorganisms including, for example, Actinomycetales, Acetobactor, Bacillales, Bacteroidales, Clostridiales, Cyanobacteria, Lactobacillales, and Proteobacteria. Preferably, the Relative Richness, which is expressed as a percentage (%), comprises Actinomycetales (order level), Acetobacter (genus level), Bacillales (species level), Bacteroidales (order level), Clostridiales (order level), and Lactobacillales (genus level) ranges from about 1 to about 98%. More preferably, the range is from about 20% to about 40%.

It is yet another object of the invention to have a metabolomics profile characterized by high resolution magic angle spinning (HRMAS) proton magnetic resonance spectroscopy (¹HMRS) of the fermentation broth. In some embodiments, the metabolomics include, e.g., one or more of acetic acid, glycolipids, lactic acid, malic acid, mycolic acid, phospholipids, polysaccharide-lipid complexes, polymixins, polyols, straight-chain fatty acids, and surfactin. Preferably, the HRMAS ¹HMRS spectrum of the fermentation broth indicates the presence of acetic acid, lactic acid, malic acid, polyols, and straight-chain fatty acids, and more preferably, acetic acid, lactic acid, malic acid, polyols, and straight-chain fatty acids in the range of about 0.5% to about 10.0%. Most preferably, the acetic acid and lactic acid are present in a range of from about 0.5% to about 4.0%.

In one embodiment, the composition contains Lactic Acid Bacteria counts of from about 1.0E+1 to about 1.0E+6. Preferably, the composition includes Lactic Acid Bacteria counts of from about 1.0E+5 to about 8.0E+5 CFU per milliliter. More preferably, the composition includes Lactic Acid Bacteria counts of from about 2.50E+5 to about 6.0E+5 CFU per milliliter. Most preferably, the composition includes a total number of microorganisms of about 3.0E+5 CFU per milliliter. The final product has a pH value of <4.0 with activity in pH range from 2 to 12, and a percent total acidity (e.g., Lactic Acid meq) between about 0.6 and about 3.2. The Emulsification Index values of the composition are preferably between about 40% and about 75%. In some embodiments, the percent total acidity means the percent lactic acid (w/v) determined according to equation 1:

% lactic acid=(mL NaOH×N NaOH×milliequivalent weight of lactic acid)×100/total volume in mL Eq. 1:

In one embodiment, the composition is essentially free of purple sulfur bacteria. In another preferred embodiment, the composition contains purple sulfur bacteria. In one embodiment, the composition contains purple sulfur bacteria at a level of from about 1.0E+1 to about 1.0E+5, and preferably, from about between 1.0E+1 to 1.0E+4.

In one embodiment, the composition is essentially free of pathogenic or putrefactive microorganisms. Preferably, the composition is essentially free of pathogenic mold and total coliform bacteria. Most preferably, pathogenic mold and total coliform are undetectable in the composition.

In some embodiments of the present invention, the compositions are hydrotropes and possess the ability to increase solubility of sparingly-soluble molecules in water. They may include some hydrophilic and lipophilic fractions, like typical surfactants, but they contain a very small hydrophobic fraction that does not allow the formation of aggregates such as micelles. As such, these are improved formulations compared to previous formulations. The final bio-degradable composition used in place of or in addition to conventional leather production chemicals and compositions, comprises not only biochemical/metabolite fermentation broth, but also viable probiotic microorganisms with a defined profile based on metagenomics and metabolomics analysis (i.e., “fingerprint”).

In yet another object of the invention, the probiotic composition is used at various stages of leather production, including raw hide preservation, pre-soaking, soaking, unhairng/liming, de-liming, bating, degreasing, tanning, wetting back, retanning, and/or fat liquoring. Use of the probiotic composition in one or more of these stages eliminates or reduces the need for use of toxic chemicals such as bacteriocides, fungicides, enzymes, surfactants, sodium sulfide, chromium, and/or synthetic fats. Exemplary applications include in beam house operations and wet-end applications.

In some aspects, biocidal chemicals such as potassium dimethyldithiocarbamate, and tetrahydro-3,5-dimethyl-2H-1,3,5-thiadiazine-2-thione typically used in the tanning process are not needed when any of the final products prepared according to the invention are applied during any stage of the leather tanning process, such as pre-soaking and/or soaking. Thus, methods according to embodiments of the invention preferably exclude such chemicals.

In an embodiment of the invention, sodium sulfide for removal of the hair during un-hairing/liming is substantially reduced and/or eliminated. Similarly, chrome 3 for tanning/retanning and/or synthetic polymer fats for fatliquoring, which are typically used in the tanning processes, are not needed and are preferably excluded or reduced, when the inventive composition is applied in the leather production process.

In embodiments of the invention, the bio-degradable probiotic composition is effective at a ratio of between 0.1 parts to 6 parts by weight dissolved in 100 litres of water.

In yet another embodiment of the invention, the exceeded amount of chrome 6 in the resulting leather article can be reduced to chrome 3 before and/or after dyeing.

In still another embodiment of the invention, the probiotic composition reduces the carbon footprint to the environment because it has better life cycle analysis (LCA) than traditional chemicals based on three categories: material consumption, energy consumption, and emission of toxic substances.

### BRIEF DESCRIPTION OF THE DRAWINGS

Drawings form part of the present specification and are included to further demonstrate certain aspects of the present invention. The invention may be better understood by reference to one or more of these drawings in combination with the detailed description of specific embodiments presented herein.

FIG. 1 is a flow diagram of the processing steps generally followed during a traditional chrome tanning process;

FIG. 2 is a photograph of the physical appearance of a wet blue hide;

FIG. 3 is a photograph of the physical appearance of the hide after unhairing/liming with the probiotic composition;

FIG. 4A illustrates the analysis of metagenomic fingerprinting profile—proportion of Relative Richness from the fermentation broth samples (6 unique samples);

FIG. 4B contains a color-coded list of the microbial community found from analysis of the fermentation broth samples (6 unique samples);

FIG. 5 is a photograph comparing the physical appearance of crust leathers from retanning using standard chemical and probiotic biochemicals;

FIG. 6 is a photograph comparing crust leathers from dyeing using standard chemicals and probiotic biochemicals;

FIG. 7 is a photograph comparing color of crust leathers cross-section observation after dyeing using standard chemicals and probiotic biochemicals;

FIG. 8 is a photograph comparing finished leathers after dyeing using standard chemicals and probiotic biochemicals;

FIG. 9 is a photograph showing raw hides contaminated with red bacteria;

FIG. 10 is a photograph showing raw hides after soaking with probiotic biochemicals;

FIG. 11 is a photograph showing wet blue processed from contaminated bacteria raw hide with probiotic biochemicals without bacteriocides;

FIG. 12 is a MALDI-TOF profile of probiotic wetting agent composition as analyzed by Voyager DE STR MALDI-TOF MS system, Applied Biosystems, using Data Acquisition software, Voyager Version 5.10.3;

FIG. 13 is a MALDI-TOF profile of probiotic dispersing agent composition as analyzed by Voyager DE STR MALDI-TOF MS system, Applied Biosystems, using Data Acquisition software, Voyager Version 5.10.3; and

FIG. 14 is a MALDI-TOF profile of probiotic solubilizing agent composition as analyzed by Voyager DE STR MALDI-TOF MS system, Applied Biosystems, using Data Acquisition software, Voyager Version 5.10.3.

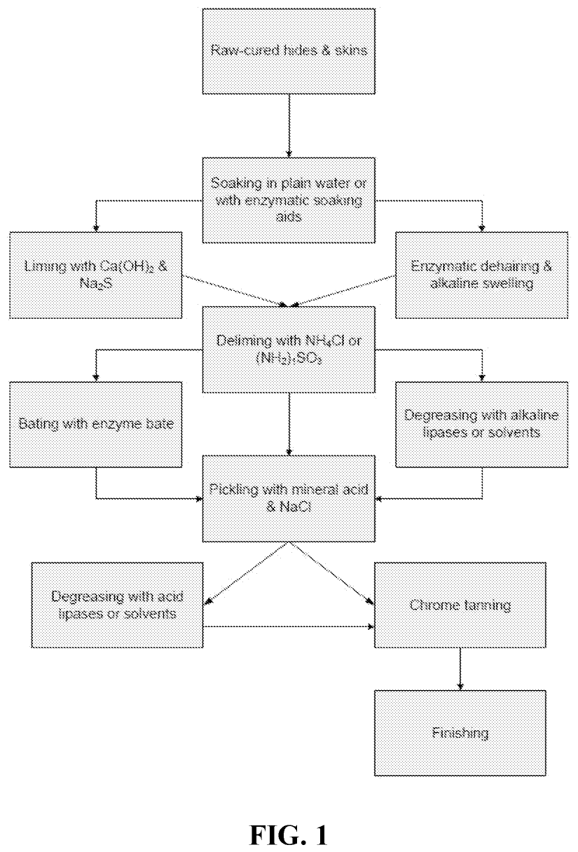

### DETAILED DESCRIPTION OF PREFERRED EMBODIMENTS

Probiotic compositions are described herein, which can be used to improve the performance of, reduce, or replace traditional chemicals used in leather production. The compositions comprise fermentation products of a microorganism consortium, and may be optionally mixed in suitable proportions with a deodorizer extracted from essential oils and/or other additives to keep the entire formulation stable over a period of one year or more. The microorganism consortium preferably comprises at least 3 microorganisms. In some embodiments, at least one microorganism is a sulfide-utilizing microorganism. In some embodiments, at least one microorganism is purple non-sulfur bacteria. Additional microorganisms include lactic acid, probiotic, and phototrophic microorganisms, as well as mixtures thereof. Microorganisms may be bacteria, yeast, fungi, and/or mold species, or mixtures thereof. Preferably, the composition includes at least one species of microorganism from lactic acid, probiotic, phototrophic, and/or sulfide-utilizing microorganism species. In another embodiment, the included sulfide-utilizing microorganism is a purple non-sulfur bacteria species.

The compositions of the present invention are hydrotropes and possess the ability to increase the solubility of sparingly-soluble molecules in water. They may include some hydrophilic and lipophilic fractions, like typical surfactants, but they contain a very small hydrophobic fraction, such that aggregates such as micelles do not form. The final bio-degradable composition can be used as a wetting agent, dispersing agent, and/or solubilizing agent. The composition can be used as a leather auxiliary agent or adjuvant, and in some embodiments, contains not only the biochemical/metabolite broth, but also includes viable probiotic microorganisms with a defined fingerprinting profile based on metagenomics and metabolomics analysis.

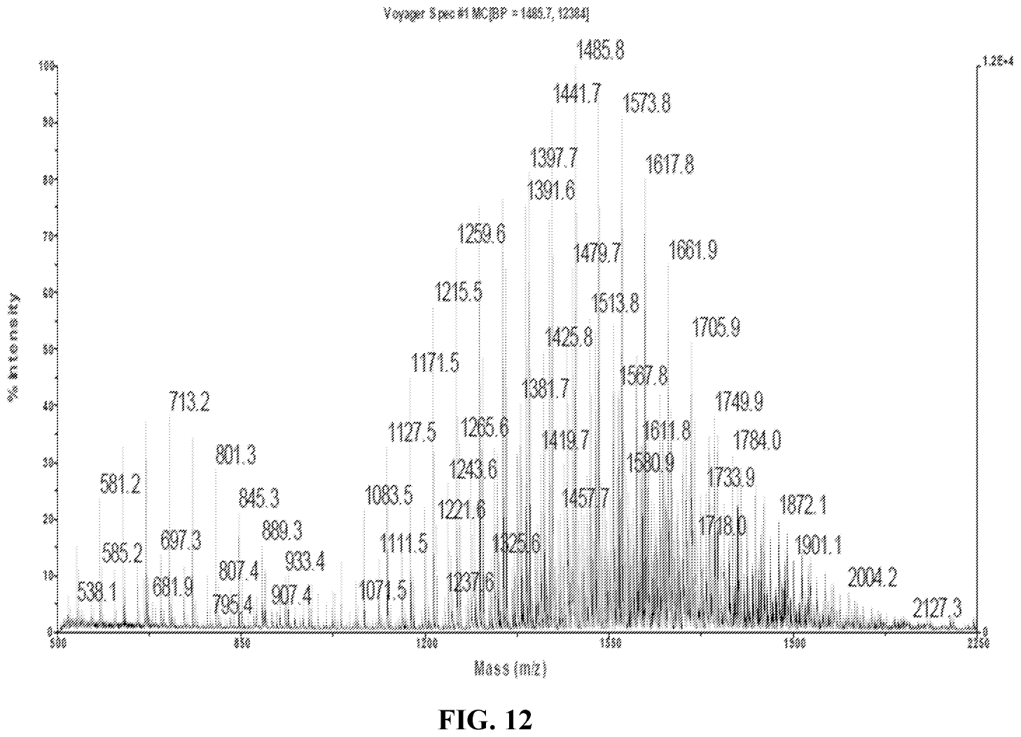

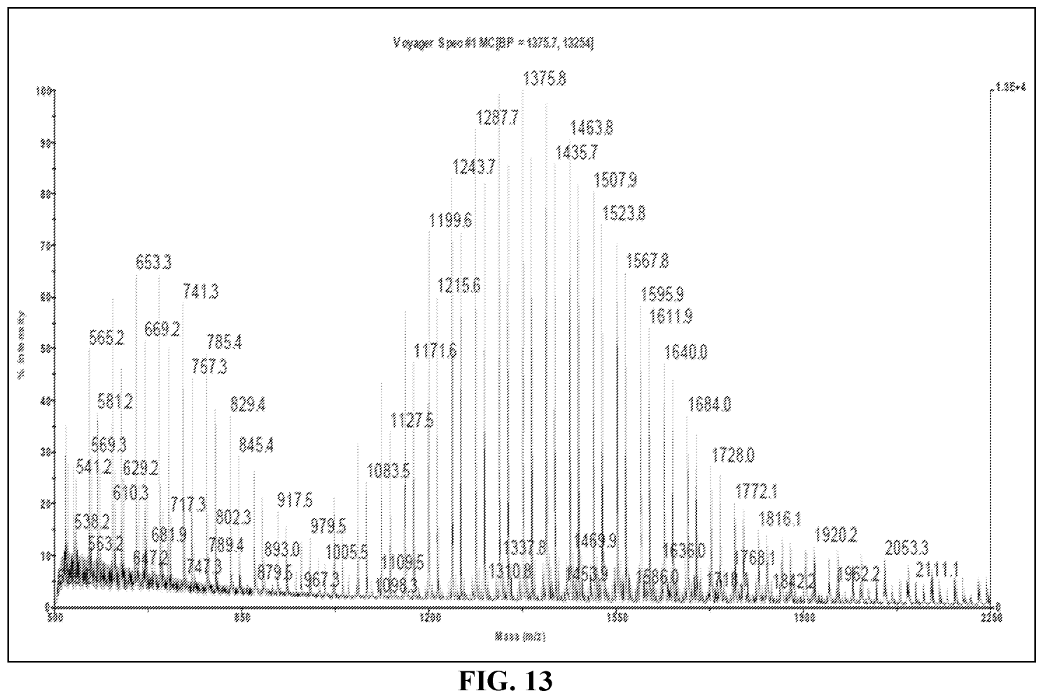

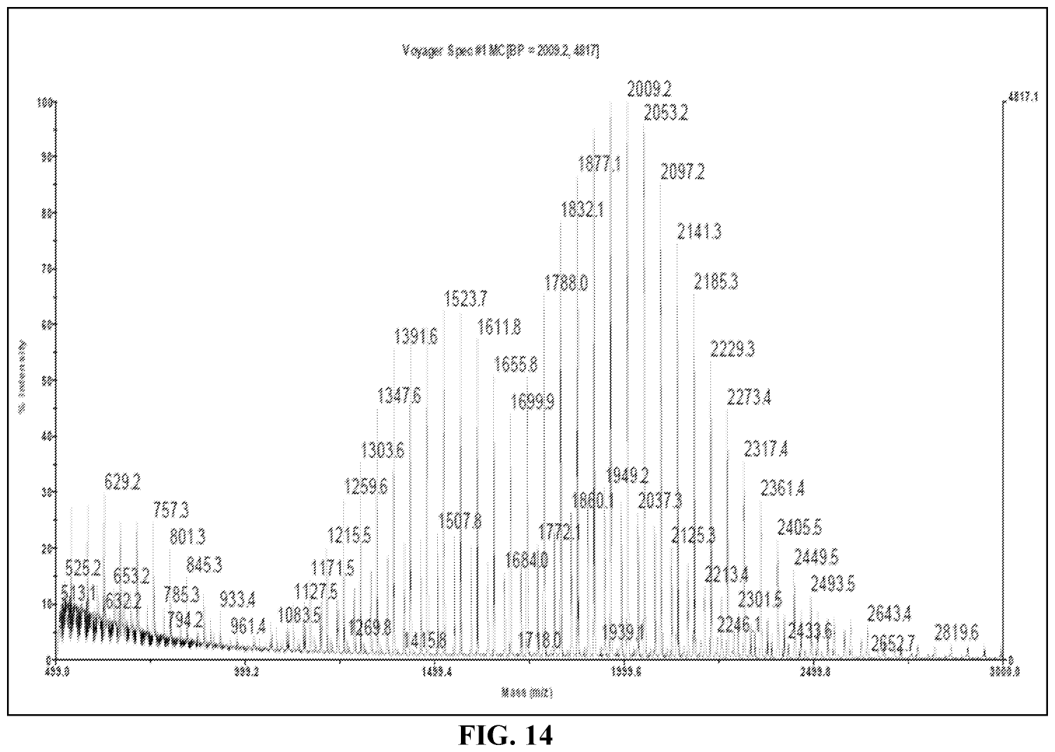

Compositions useful in this invention include microorganisms and additives. The microorganisms may include species of bacteria and fungi, including yeast and mold species. Suitable microorganisms include those commonly known in the art as phototrophic, lactic acid, probiotic, and sulfide-utilizing microorganisms. Exemplary fermentation products of the various microorganism consortiums are shown in FIGS. 12-14.

Examples of useful phototrophic, lactic acid, probiotic, and sulfide-utilizing microorganisms are found, for example, in Bergey's Manual of Determinative Bacteriology and Bergey's Manual of Systematic Bacteriology. For example, sulfide-utilizing microorganisms include species of Purple Non-sulfur Bacteria, Chromatianeae, Green Sulfur Bacteria, Colorless Sulfur Bacteria, and Filamentous Green Bacteria. Probiotic microorganisms may include Lactobacillus genus, Enterococcus genus, Bifidiobacterium genus, Bacillus genus, Pseudomonas genus, Sporolactobacillus genus, Micromonospora genus, Micrococcus genus, Rhodococcus genus, and E. coli. Phototrophic microorganisms may include Rhodopseudomonas species, Rodobactor species, and combinations thereof. For example, phototrophic microorganisms may include Rhodopseudomonas palustris, R. sphaeroides, Rhodospirillum centenum, R. photometricum, R. rubrum, Rhodopila globiformis, Rhodobacter sphaeroides, and combinations thereof. Lactic acid microorganisms may include Lactobacillus, Lactococcus and combinations thereof. For examples, lactic acid microorganisms may include Lactobacillus casei, L. plantarum, L. acidophilus, L. fermentum, L. brevis, L. lactis, L. reuteri, L. bulgaricus, L. cellobiosus, L. curvatus, L. delbrukil, L. helbeticus, L. euterii, L. salivarius, L. rhamnosus, L. gaserli, L. jensenii, L. sporogenes, Lactococcus lactis, Streptococcus (Enterococcus) faecium, S. faecalis, S. cremoris, S. diacetylactis, S. intermedius, S. lactis, S. thermophilus, Pediococuss acidilactici, P. cerevisiae (damnosus), P. pentosaceus, P. acidilacticii, Leuconostoc mesenteroides, and combinations thereof. Bacilli microorganisms may include Bacillus genus and combinations thereof. For example, Bacilli microorganisms may include Bacillus licheniformis, B. subtilus, B. toyoi, B. amyloliquefaciens, B. megateriu, B. pumilus, B. coagulans, B. lentus, B. thermophilus, B. laterosporus, B. cereus, B. circulans, and combinations thereof. Bifidobacterium microorganisms may include Bifidobacterium genus and combinations thereof. For example, Bifidobacterium microorganisms may include Bifidobacterium bifidum, B. pseudolongum, B. thermophilus, B. adolescentis, B. animalis, B. infantis, B. longum, and combinations thereof Pseudomonas microorganism may include Pseudomonas aeruginosa, P. putida, P. cepacia, P. fluorescens, and combinations thereof. Yeast microorganisms may include Saccharomyces genus and combinations thereof.

In a preferred embodiment, the composition contains at least one microorganism. More preferably, the composition includes a consortium of two or more microorganisms. It is contemplated that where two or more microorganisms form the composition, the microorganisms are co-cultured. The microorganisms may be propagated by methods known in the art. For example, the microorganisms may be propagated in a liquid medium under anaerobic or aerobic conditions. Suitable liquid mediums used for growing microorganism include those known in the art.

In one aspect, the composition (with or without additives) includes a total number of microorganisms of about 1 to about 1 million colony forming units (CFU) per milliliter. Preferably, the composition includes a total number of microorganisms of about 100,000 to about 800,000 CFU per milliliter. More preferably, the composition includes a total number of microorganisms of about 250,000 to about 600,000 CFU per milliliter. Most preferably, the composition includes a total number of microorganisms of about 300,000 CFU per milliliter.

In one aspect, the microorganisms present in the composition may be living or non-living. In another aspect, the composition includes both living and non-living microorganisms. Compositions containing non-living microorganisms may contain extracts of the microorganisms in addition to or in lieu of the microorganism itself. Such extracts may be considered a liquid fermentation product of the living microorganisms. The extracts of microorganisms include, by way of example, organic acids such as acetic acid, lactic acid, and/or fatty acids, small chain peptides, biopolymers, and other compounds such as vitamins and polyols that are produced by microorganisms and are capable of eliciting an effect on an environment regardless of the living status of the microorganism.

In some embodiments, the compositions may also include additives. Suitable additives include substances known in the art that may support growth, production of specific metabolites by the microorganism, alter pH, enrich for target metabolites, and combinations thereof. Exemplary additives include carbon sources (e.g., sugars, molasses, rum, starch etc.), nitrogen sources, inorganic salt, organic acid, growth media, vitamins, minerals, acetic acid, amino acids and the like.

In yet another aspect, the present invention provides one or more compositions mixed in suitable proportions with a deodorizer extracted from essential oils and/or other additives to keep the entire formulation stable over a period of one year or more. In one or more embodiments, a deodorizer extracted from essential oils and other additives are included in a range of from about 0.5 to about 3% by weight of the total volume (w/v) of fermentation broth to yield the final product.

One or more embodiments of the final products of the invention have a metagemonics profile characterized by % of Relative Richness of microorganisms such as Actinomycetales, Acetobactor, Bacillales, Bacteroidales, Clostridiales, Cyanobacteria, Lactobacillales, and Proteobacteria. Preferably, the Relative Richness percentage comprises Actinomycetales (order level), Acetobacter (genus level), Bacillales (species level), Bacteroidales (order level), Clostridiales (order level), and Lactobacillales (genus level) in a range of about 1 to about 98%. More preferably, the range is about 20% to about 40%.

Some embodiments of the invention have a metabolomics profile characterized by HRMAS ¹HMRS spectrum of the fermentation broth indicating the presence of metabolites, such as acetic acid, glycolipids, lactic acid, malic acid, mycolic acid, phospholipids, polysaccharide-lipid complexes, polymixins, polyols, straight-chain fatty acids, and/or surfactin. Preferably, the HRMAS ¹HMRS spectrum of the fermentation broth indicates the presence of acetic acid, lactic acid, malic acid, polyols, straight-chain fatty acids. More preferably, acetic acid, lactic acid, malic acid, polyols, and/or straight-chain fatty acids are present in a range of about 0.5% to about 10.0%. Most preferably, acetic acid and lactic acid are present in a range of about 0.5% to about 4.0%.

In one embodiment, the composition contains Lactic Acid Bacteria counts between about 1.0E+1 to about 1.0E+6. Preferably, the composition includes Lactic Acid Bacteria counts of about 1.0E+5 to about 8.0E+5 CFU per milliliter. More preferably, the composition includes Lactic Acid Bacteria counts of about 2.50E+5 to about 6.0E+5 CFU per milliliter. Most preferably, the composition includes a total number of microorganisms of about 3.0E+5 CFU per milliliter.

In one embodiment, the composition is essentially free of purple sulfur bacteria. In another preferred embodiment, the composition contains purple sulfur bacteria. In one embodiment, the composition contains purple sulfur bacteria at a level of from about 1.0E+1 to about 1.0E+5 CFU/mL, and preferably, from about 1.0E+1 to about 1.0E+4 CFU/mL.

In one embodiment, the composition is essentially free of pathogenic or putrefactive microorganisms. Preferably, the composition is essentially free of pathogenic Mold and Total Coliform. Most preferably, pathogenic Mold and Total Coliform are not present in detectable levels in the composition.

The probiotic composition is used at various stages of leather tanning including raw hide preservation, pre-soaking, soaking, liming, de-liming, bating, degreasing, tanning, wetting back, retanning, fat liquoring, eliminating or reducing the need for use of toxic chemicals such as bacteriocides, fungicides, enzymes, surfactants, sodium sulfide, chromium, and/or synthetic fats.

In some aspects, biocidal chemicals such as potassium dimethyldithiocarbamate, and tetrahydro-3,5-dimethyl-2H-1,3,5-thiadiazine-2-thione typically used in the tanning process are not needed when any of the invented products is applied in the leather tanning process such as pre-soaking and/or soaking.

In an embodiment of the invention sodium sulfide for removal of the hair during un-hairing/liming is substantially reduced and/or eliminated. Similarly, chrome 3 for tanning/retanning and/or synthetic polymer fats for fatliquoring, which are typically used in the tanning processes, are not needed, and are preferably excluded and/or are reduced, when the invented composition is applied in the leather production process.

In embodiments of the invention, the bio-degradable probiotic composition is effective at a ratio of between about 0.1 parts to about 6 parts by weight dissolved in 100 litres of water.

In yet another embodiment of the invention the exceeded amount of chrome 6 in leather article can be reduced to chrome 3 before or after dyeing.

In still another object of the invention the probiotic composition reduces the carbon footprint in the environment because it has better LCA than traditional chemicals based on three categories; material consumption, energy consumption and emission of toxic substances.

The compositions of the invention may be in liquid or dry form. The composition may comprise an aqueous suspension. This aqueous suspension may be provided as a concentrated stock solution which is diluted prior to application or as a diluted solution ready-to-use. Also, the composition may be a wettable powder, granules, dust, pellet, or colloidal concentrate. Such dry forms may be formulated to dissolve immediately upon wetting with water or suitable solvent system, or dissolve in a controlled-release, sustained-release, or other time-dependent manner. Also, the composition may be in a dry form that does not depend upon wetting or dissolving to be effective.

The compositions may additionally be provided in a formulation capable of spray. Spray compositions may include surfactants, emulsifiers, and wetting agents. The spray may be a liquid or an aerosol. The compositions of the present invention are stable under various conditions as a liquid or dry form. Preferably, the compositions of the present invention are stable at room temperature.

The resulting biodegradable/probiotic compositions can be formulated for use as a wetting agent, dispersing agent, and/or solubilizing agent, and are useful as partial or complete chemical replacements or as adjuvants in leather production processes. For example, different additives can be included with the fermentation broth to yield different properties in the final product (that is used in leather production). In addition, different manufacturing protocols may be used to alter the properties of the final product, such as mixing conditions. The resulting compositions have different HLB values (Hydrophile-Lipophile Balance) due to different physico-chemical properties. Probiotic wetting agent compositions according to embodiments of the invention have medium relative HLB values (e.g., at least 7, in some cases from 7 to about 10). Probiotic dispersing agent compositions according to embodiments of the invention likewise have medium relative HLB values (e.g., at least 7, in some cases from 7 to about 10). As used herein “wetting” and “dispersing,” respectively refer to surface wetting (two dimensional) versus dispersion in three dimensions, and could have the similar HLB values. Probiotic solubilizing agent compositions according to the invention have higher HLB values relative to the wetting and dispersing agents (e.g., greater than about 13, and in some cases from about 13 to about 18). Probiotic solubilizing agent compositions can be used to degrease very fatty animal hides. Likewise, probiotic wetting agent and/or dispersing agent compositions can be initially used for not-so fatty animal hides. Similarly, although the dispersing agent may find use in finishing stages of the leather production process, the wetting agent is generally limited to initial processing stages.

Thus, use of the subject composition(s) in various applications will benefit an environment that would benefit from a microorganism composition. In one aspect, the invention therefore provides a method of protecting or improving the environment by using the subject composition(s) in various applications that affect the environment. In one embodiment, the method includes the use of the subject composition(s) to augment, replace and/or reduce the need for chemical compositions, such as chemicals used in leather tanning.

The subject compositions disclosed herein are useful as a chemical replacement and/or adjuvant. The present invention encompasses methods of benefiting an environment that would benefit from a microorganism composition by using the subject composition(s) to augment, reduce and/or replace chemical compositions, such as chemicals used in leather production, including tanning operations.

Generally, the process of tanning leather involves many different toxic chemicals, to tan rawhide into high quality leather product. Consequently, the leather processing industry is one of the most polluting and worst offenders of the environment.

Improved methods of leather production, including leather tanning processes, are contemplated that use the composition(s) of the invention. In particular, methods of leather production that replace a chemical typically used in the production process with a composition of the invention are contemplated. The composition(s) described above are biodegradable and can be used for replacing chemical biocides as well as in conjunction with soaking auxiliary agents during tanning soaking operations.

Leather production typically includes prepping a hide, removal of keratinous matter, removal of natural grease and fats, batting, and eventually tanning. During this process the compositions of the invention may be used to replace chemicals typically used, or simultaneously, to enhance the effects of such chemicals. One skilled in the art will appreciate that the use of compositions of the invention will depend upon the desired source of hide being tanned, the method of tanning, and the desired end-product. For instance, tanning resulting in a more flexible hide, which may be desired for luggage and furniture, whereas a more stretchable hide may be desired for handbags and clothing.

The hide may be obtained from any animal including, for example, farm animals, game and the like. Specific animals include, without limitation, alligators, cattle, crocodile, deer, elk, goats, kangaroos, ostriches, oxen, pig, sheep, snakes, oxen, and/or yak. The hide may consist of both the top grain layer and the split leather or either layer separately.

In some aspects, the probiotic composition is provided alone as a replacement for chemicals typically used in the tanning process. Examples include potassium dimethyldithiocarbamate, and tetrahydro-3,5-dimethyl-2H-1,3,5-thiadiazine-2-thione, essentially used as a bactericide. In other aspects, the probiotic composition is provided in combination with another tanning agent. Examples of tanning agents that may be provided with the present invention include tannins, chromium tanning reagents, and aluminum tanning reagents, as well as others known in the art.

The methods comprise contacting an animal hide with a composition of the invention. Contacting can be accomplished in a variety of ways. In some embodiments, the hide is submerged in the liquid formulation, in other aspects the dry formulations are wetted to produce a paste, which is applied as a coating onto the hide. The formulation may also be sprayed onto the hide. In some embodiments, several hides are submerged in a liquid formulation containing the present invention in a rotating drum.

The amount of time that the composition is contacted with the animal hide can and will vary depending on the type of hide being contacted and the desired qualities of the resulting tanned hide, including the amount of penetration desired. In some aspects, the hide is contacted with the composition for a period ranging between about 1 hour to 10 days. In some aspects, the process is repeated one or more times to achieve the desired tanning.

Stages of leather production wherein bio-degradable probiotic compositions of present invention are employed include:

1. Soaking: Dosing ranges may depend on raw hides and skins origins, breed, genre, season, preservation method and conditions, and tannery equipment. Suggested usages are as the follows:
Washing: about 1 to about 2 g/L of probiotic wetting agent composition;
Presoaking: about 3 to about 5 g/L of probiotic dispersing agent composition in combination with 0 to about 1 g/L of probiotic solubilizing agent composition;
Main soaking: about 3 to about 5 g/L of probiotic wetting agent composition in combination with 0 to about 3 g/L of probiotic solubilizing agent composition.

2. Liming: To help lime dispersion and penetration into the collagen fibres. A regular swelling is achieved, with less wrinkles. Dosing about 2 g/L of probiotic dispersing agent composition at the beginning of unhairing-liming process.

3. De-liming: Dosing up to about 3 g/L of probiotic solubilizing agent composition at the latest washes to neutralize lime and disperse and eliminate remaining fats from mechanical degreasing (fleshing).

4. Bating: Dosing up to about 2 g/L of probiotic solubilizing agent composition for degreasing and cleaning grain side of hides and skins.

5. Degreasing: for skins with very high amount of fat like sheep or pig skins. Dosing about 3 to about 5 g/L of probiotic solubilizing agent composition, when skins are pickled or after depickling in combination with a anionic or non-ionic surfactant agent with good emulsifying properties. Normally, operation is repeated twice or three times.

6. Tanning:.
Chrome: Dosing about 2 to about 3 g/L of probiotic dispersing agent composition before the addition of the chromium tanning salts.
White: Dosing about 3 to about 5 g/L of the probiotic composition before the tanning agent (glutaraldehyde or other organic compounds)
Vegetable: Dosing about 3 to about 5 g/L of the probiotic composition together with the vegetable tannin or extract.

7. Washing/wetting back: Dosing about 1% to about 2% on leather shaved weight (wet-blue, wet-white or vegetable leather) of the novel composition as a dispersing, wetting or to solubilizing agent depending on leather condition) or about 2% to about 4% on dried weight if leather are dried after tanning.

8. Retanning/fat liquoring: Dosing half of main retanning or fatliquor before or during the retanning or fat liquoring operation.

9. Dyeing: Dosing half of dye for dark or medium shades or same amount of dye for pastel shades before or together with the dye in the main or top dyeing.

10. Finishing: the probiotic composition can help to reduce or eliminate solvents in some coating applications like: dosing about 20 to about 200 g/L of coating solution.

11. Spray dyeing/pigmenting: the probiotic composition can be used with finishing dyes to adjust the shades and enhancing the final appearance of leather articles by improving the intensity and brightness of the colours. As probiotic compositions do not contain VOC, they are an environmental solution for restricted solvents, especially suitable in high performing articles like automotive and aviation leather. Probiotic composition does not leave a harsh feel on the surface finished leather like solvents.

12. Impregnation: the probiotic composition is suitable to be used as penetrating agent of impregnation of leather finishing coats. They do not affect the physical properties of flex resistance and adhesion of the finishing layers. Therefore, solvents which involve VOCs are not necessary.

The composition(s) of the subject invention can also be used to reduce the amount of sodium sulfide needed to remove hair from the article during dehairing/liming process. The composition can also reduce chromium contamination in the effluent water from the production process. Due to hydrotropic properties of the biodegradable composition, it allows water molecules to penetrate the protein chains. When water molecules are in contact with protein structure, hydrogen bonds are broken. The trivalent chromium reacts with the free carboxyl groups and, initially, complexes of Cr are formed, crosslinking protein chains: Protein-CO—O—Cr(OH)—SO₄—Cr(OH)—CR(OH)-o-CO-Protein. As a result, carboxyl groups are free, Cr then has more functional groups to be fixed to, thus better exhaustion of the Cr with the leather, allowing the reduction of the trivalent chromium used in tanning as well as reduction of the hexavalent chromium formed through oxidation. This, in turn, results in lower Cr contamination in the effluents.

In yet another embodiment, the bio-degradable composition can be used to reverse the hexavalent chromium found in a leather article before and/or after dyeing. In many instances, leather articles are rejected and/or then destroyed due to excessive amounts of hexavalent chromium contamination. When the invented composition is sprayed over the contaminated leather article over a period of about 1 hour to about 12 hours. The amount of the hexavalent chromium is decreased, and found to be within acceptable limit for the finished leather article before and/or after dyeing.

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the invention. It is to be understood, however, that these examples are provided by way of illustration and nothing therein should be taken as a limitation upon the overall scope of the invention. The following examples are illustrated to show the working of the probiotic composition compared to its chemical equivalents

### Example 1

### Use of Composition Wetting and/or Dispersing Agent in Pre-Soaking/Soaking

Results from a third party laboratory show that high microbial counts found in soaking baths containing bio-degradable composition do not adversely affect the quality of the hide after soaking or after wet blue conversion (Table 1 and 2).

TABLE 1
Evaluation of the degree of soaking efficiency
and biological analysis of soaking baths.

| | Soaking | Bacterial Count |
|---|---|---|
| Trial Run | Efficiency | (CFU/ml) |
| 1 | Good | 860,000 |
| 2 | Regular | 6,450,000 |
| 3 | Regular | 9,550,000 |
| 4 | Good | 10,800,000 |
| 5 | Good | 9,700,000 |
| 6 | Good | 11,100,000 |
| 7 | Good | 8,400,000 |
| 8 | Good | 5,800,000 |
| 9 | Good | 5,500,000 |
| 10 | Good | 500,000 |
| 11 | Good | 17,200,000 |
| 12 | Good | 22,700,000 |
| 13 | Good | 600,000 |
| 14 | Good | 900,000 |
| 15 | Good | 14,500,000 |
| 16 | Good | 8,400,000 |
| 17 | Good | 7,700,000 |
| 18 | Good | 13,900,000 |
| 19 | Good | 9,800,000 |
| 20 | Good | 4,500,000 |

TABLE 2
Evaluation of Chrome Tanned Leather and Chemical Analyses

| | |
|---|---|
| Degree of | Chemical Analysis |

| Trial Run | Tanning | pH | % Chromium Oxide | % Fat |
|---|---|---|---|---|
| 1 | Good | 3.86 | 3.2 | 1.1 |
| 2 | Good | 3.49 | 3.5 | 1.9 |
| 3 | Good | 3.54 | 5.0 | 1.1 |
| 4 | Good | 3.41 | 3.3 | 1.2 |
| 5 | Good | 3.44 | 3.4 | 1.3 |
| 6 | Good | 3.54 | 3.3 | 1.3 |
| 7 | Good | 3.43 | 3.9 | 0.8 |
| 8 | Good | 3.62 | 3.8 | 0.7 |
| 9 | Good | 3.43 | 4.1 | 0.8 |
| 10 | Good | 3.58 | 2.9 | 0.9 |
| 11 | Good | 3.48 | 3.4 | 0.8 |
| 12 | Good | 3.42 | 3.1 | 0.9 |
| 13 | Good | 3.44 | 3.3 | 0.7 |
| 14 | Good | 3.47 | 3.3 | 0.9 |
| 15 | Good | 3.50 | 4.2 | 0.5 |
| 16 | Good | 3.55 | 4.3 | 0.6 |
| 17 | Good | 3.65 | 4.4 | 0.7 |
| 18 | Good | 3.34 | 2.9 | 1.2 |
| 19 | Good | 3.34 | 3.3 | 1.6 |
| 20 | Good | 3.38 | 3.4 | 1.0 |

To further emphasize the outcomes, blind sample trials with the highest amount of bacterial counts in soaking baths, Trial 12 (22.7×10⁷ CFU/ml), were compared to the lowest amount of bacterial counts in soaking baths, Trial 10 (5.0×10⁵ CFU/ml), and there was no sign of hide damage caused by bacterial attack after soaking and after wet blue conversion (Table 2). In addition, the quality of the wet blues from Trial 12 and Trial 10 are equal (Table 2). There were 20 blind testing trials in total.

In each trial run, the raw hide was prewashed for 30 minutes with the probiotic composition. Subsequently, it was subjected to a soaking process with the novel wetting and/or dispersing agent for up to 8 hours. Table 1 indicates the degree of soaking efficiency as well as bacterial load data. Under normal conditions, it would be recommended that a bactericide product should be used since all bacterial counts in soaking exceed their limit of 2.0×10⁵ CFU (Table 1). However, in the presence of the probiotic composition without the use of bactericides such as Potassium dimethyldithiocarbamate, and Tetrahydro-3,5-dimethyl-2H-1,3,5-thiadiazine-2-thione, very good results were achieved.

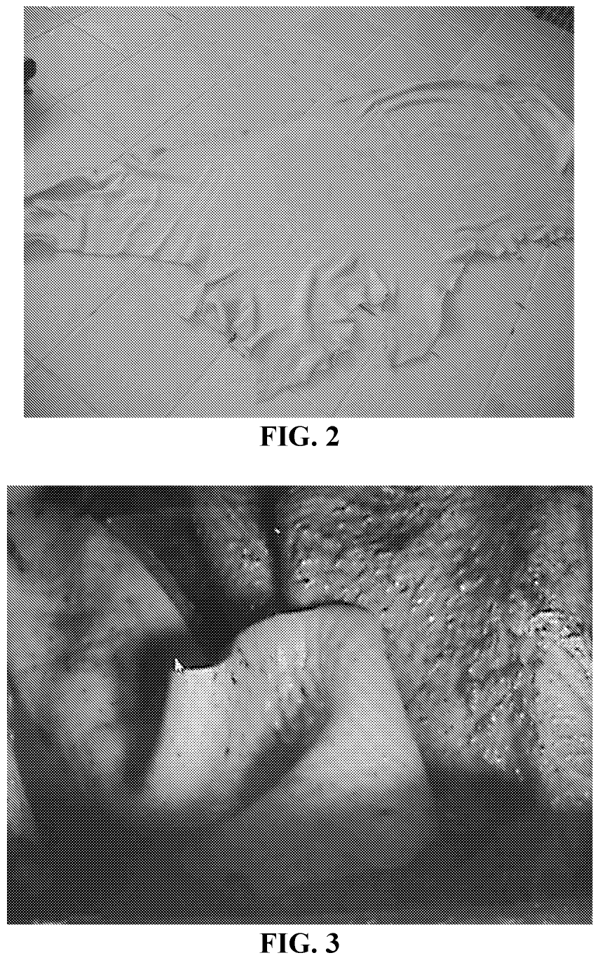

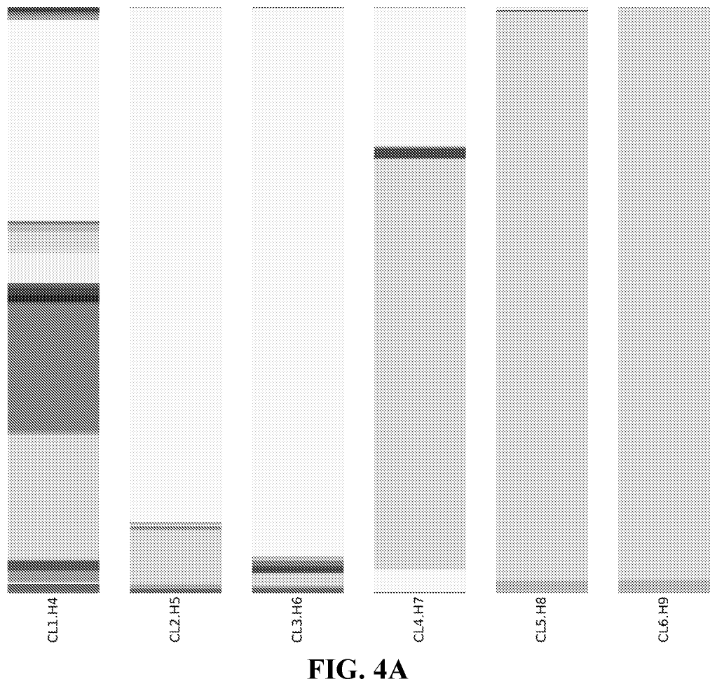

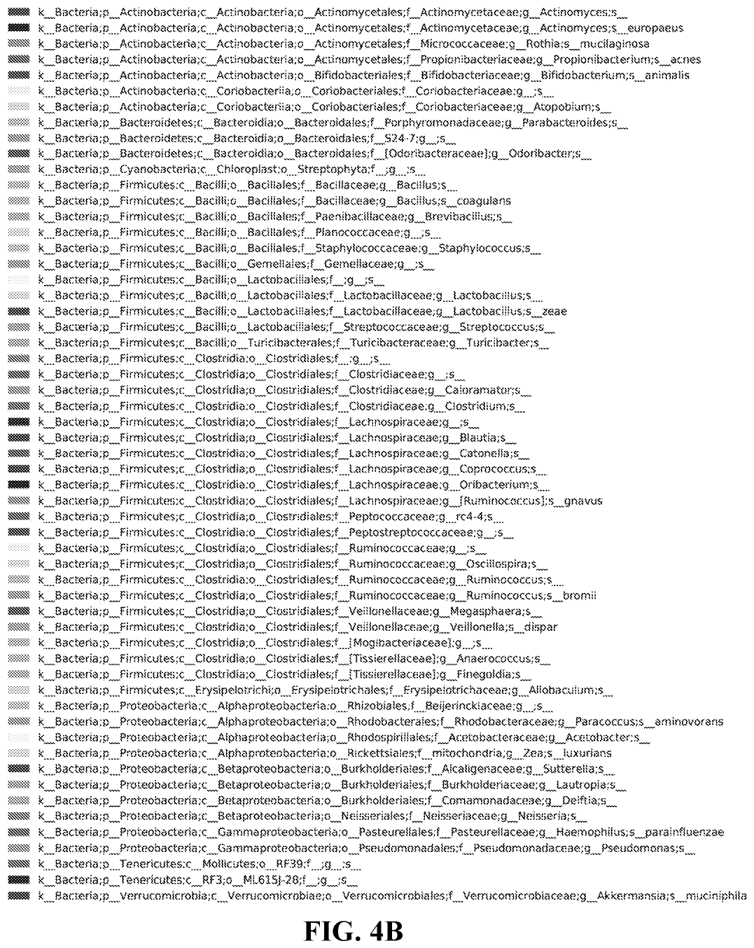

All hides from the soaking process were then processed to wet blues. All processed wet blues had a good degree of tanning and showed no problem with shrinkage or with boiling tests. The pH, % chromium oxide and fat contents were within the recommended range (Table 2). Likewise, through visual inspection, all wet blues were normal and acceptable to standards (FIG. 2).

### Example 2

### Use of Composition as Dispersing/Solubilizing Agent in Unhairing/Liming

An industrial 4,000 Kgs capacity drum was used in the experiment. Eight pieces of raw hides each weighing 200 kg were used.

In pre-washing, 200 liters of water, 0.2% of the probiotic composition was used. Additional 200 liters of fresh water and 0.5% of probiotic biochemicals were used in soaking. Unlike the traditional process, the addition of bactericide, enzyme and/or degreaser was used needed for the probiotic treatment. The soaked water was reused plus 0.3% of probiotic biochemicals and 0.84% of sodium sulfide (Table 3). The amount of lime was the same between traditional process and the process with probiotic biochemicals.

In conclusion, it was found that the total process time of the experiment could be reduced by 28% (Table 3). More importantly, the amount of sodium sulfide needed for de-hairing was reduced by 30% without negatively impacting the overall quality of the tested article.

TABLE 3

| | Process time (hours) | Process time (hours) |
|---|---|---|
| | Traditional | With Probiotic Biosurfactants |
| Pre-washing | 4 | 0.5 |
| Soaking | 1 | 4 |
| Liming* | 4 | 2 |

*With 1.2% Sodium sulfide (traditional) and 0.84% Sodium sulfide (with probiotic composition), respectively.

In another example (Table 4), the experiment was done using 4 raw hides each weighed 100 Kgs. In pre-washing stage, 100 liters of water, plus 0.2% of the probiotic biochemicals were used.

TABLE 4

| | Process time (hours) | Process time (hours) |
|---|---|---|
| | Traditional | With Probiotic Biosurfactants |
| Pre-washing | 1 | 0.5 |
| Soaking | 24 | 4 |
| Liming* | 12 | 15 |

*With 1.4% Sodium sulfide (traditional) and 0.9% Sodium sulfide (with probiotic composition), respectively.

Prewashed water was discarded, then 100 liters of water, plus 0.5% of the probiotic biochemicals were used without the addition of bactericide, enzyme and/or chemical degreaser during soaking. The soaking water was reused plus 0.3% of the probiotic biochemicals and 0.9% of Sodium sulfide. Sodium sulfide (1.4%) was used for the traditional process. The amount of lime used was the same for both treatments.

In conclusion, it was found that the total processed time and Sodium sulfide was reduced by 47% and 35% with the probiotic treatment Table 4.

### Example 3

### Use of Composition in Fat Liquoring

The experiment was done using Spanish merino wool wet blue. Eight wet blue article with the total weight of 8.5 Kgs were used using a pilot plant drums. There was no improvement on degreasing effect. However, by replacing traditional surfactant (Borron SAF) with the probiotic composition, the total amount of fatliquor could be reduced by 10%. Most fatliquors are manufactured from polymers, which is known to have high carbon footprint according life cycle assessment (LCA) standard unlike the probiotic composition.

During the soaking steps of a trial vegetable tanning process, raw calf hide was soaked in a drum containing the probiotic composition or the control solution (chemical surfactant). In particular, one drum of 105 kgs of buff calf wet salted hide was processed using the Tanning Formulation. The probiotic composition was applied at 0.2% by weight of pelt weight during the soaking stage, with no added preservatives. In a parallel process, one drum of 1100 kgs of buff calf wet salted hide was processed using the control formulation. The resultant tanned products were compared in regard to color, growth and drawn marks, grain, and flanks (Table 5). The hide tanned using the probiotic composition exhibited uniform color, a grade 2 rating for growth and drawn marks, a close and fine grain, and well-filled flanks. In comparison, the hide tanned using the control solution exhibited uniform color, a grade 6 rating for growth and drawn marks, a coarse grain, and loose and baggy flanks.

TABLE 5

| | | | Time | |
|---|---|---|---|---|
| | | | (min- | |
| Process | % | Product | utes) | Comments |
| Fatli- | 600 | Water | | |
| quoring | 1.8 | Eskatan GLH | | Synthetic Sulfite ester |
| | 1.8 | Fatliquor NL | | |
| | 1.8 | Fatliquor ET | | Reduction of 10% |
| | | | | from control |
| | 0.5 | Probiotic | | Control Borron SAF |
| | 0.15 | Bactericide | 5 | |
| | 0.005 | Antistatic | 55 | |
| | 0.2 | Formic acid (85%) | 20 | |
| | 0.2 | Formic acid (85%) | 40 | |

During the soaking steps of a trial chrome tanning process, raw bovine hide was soaked in a drum containing the probiotic composition or the control solution. In particular, two drums containing 1400 kgs of bovine wet salted hide was processed using the probiotic composition. The probiotic composition was applied at 0.3% by weight of pelt weight during the soaking stage and at 0.15% of pelt weight before the chrome stage with no added preservatives. In a parallel process, two drums of 110 kgs of bovine wet salted hide were processed using the control solution. The resultant tanned products were compared in regard to color, growth and drawn marks, and feel and odor (Table 6, 7, and 8). The hide tanned using the Tanning Formulation exhibited uniform color, a grade 3 rating for growth and drawn marks, no irritation upon hand feel, and no odor. In comparison, the hide tanned using the control solution exhibited uniform color with blood-stains, a grade 7 for growth and drawn marks, itchy skin upon hand feel, and a fungicide odor. The hides tanned with the probiotic composition had reduced growth mark and drawn grain and better cleaning of the grain and flesh sides of the hide, thus, resulting in improved leather quality. Also, the probiotic composition is completely biodegradable and, therefore, reduces load on the effluent.

TABLE 6
Probiotic composition Trial in Chrome Tanning.

| | Probiotic | |
|---|---|---|
| | composition Lot | Control Lot |
| Color | Uniform blue | Uniform blue, but with |
| | | blood stains |
| Growth & Drawn Marks* | Grade 3 | Grade 6 |
| Hand Feel | No irritation | Itchy skin |
| Odor | No Odor | Fungicide odor |

*Grade 1 being best and Grade 10 being worst

TABLE 7
Probiotic composition Trial In Vegetable Tanning.

| | Probiotic | |
|---|---|---|
| | composition Lot | Control Lot |
| Color | Uniform beige | Uniform beige |
| Growth & Drawn Marks* | Grade 2 | Grade 6 |
| Grain | Close & Fine | Coarse |
| Flanks | Well Filled | Loose & Baggy |

*Grade 1 being best and Grade 10 being worst

TABLE 8
Probiotic composition Trial.

| | Probiotic | |
|---|---|---|
| | composition Lot | Control Lot |
| Color | Uniform blue | Uniform blue, but with |
| | | blood stains |
| Growth & Drawn Marks* | Grade 3 | Grade 7 |
| Hand Feel | No irritation | Itchy skin |
| Odor | No Odor | Fungicide odor |

*Grade 1 being best and Grade 10 being worst

### Example 4

### Improve Dispersion of Natural Fat, Fatliquors and Dyes by Replacing Chemical Surfactants by Probiotic Biochemicals

Material: 4 heavy bovine hides, wet-blue, shaved to 1.4-1.6 mm. Total wet-blue weight 49 KG.

Article: Furniture upholstery

Process:

Wash: 0.2% probiotic composition replaced chemical surfactant based on 0.2 fatty alcohol ethoxylated and 0.1% oxalic acid.

Neutralizing, fatliquoring, retanning: followed tannery standard recipe.

Leathers went to samming and drying.

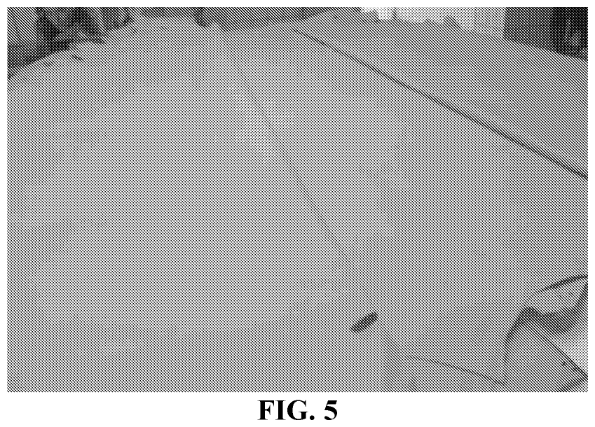

Next day crust leathers were checked. No visual differences were appreciated with standard (FIG. 5, standard in the middle). Light fastness >3, within specifications.

Crust leather was weighted (25 KG) and loaded to dyeing drums.

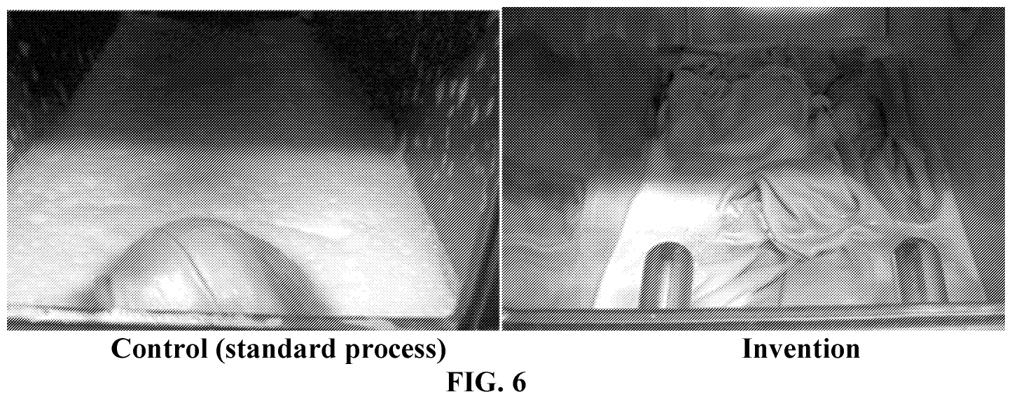

Wetting back: 4% Probiotic compounds replacing 1% ammonia and 3% surfactant. pH after wetting back was 4 instead standard 6.3. Much less foam was observed (FIG. 6, right probiotic compounds, left standard).

Dyeing: 2% Probiotic compound replaced 1% ammonia and 2% dye auxiliary based on fatty amine ethoxylated.

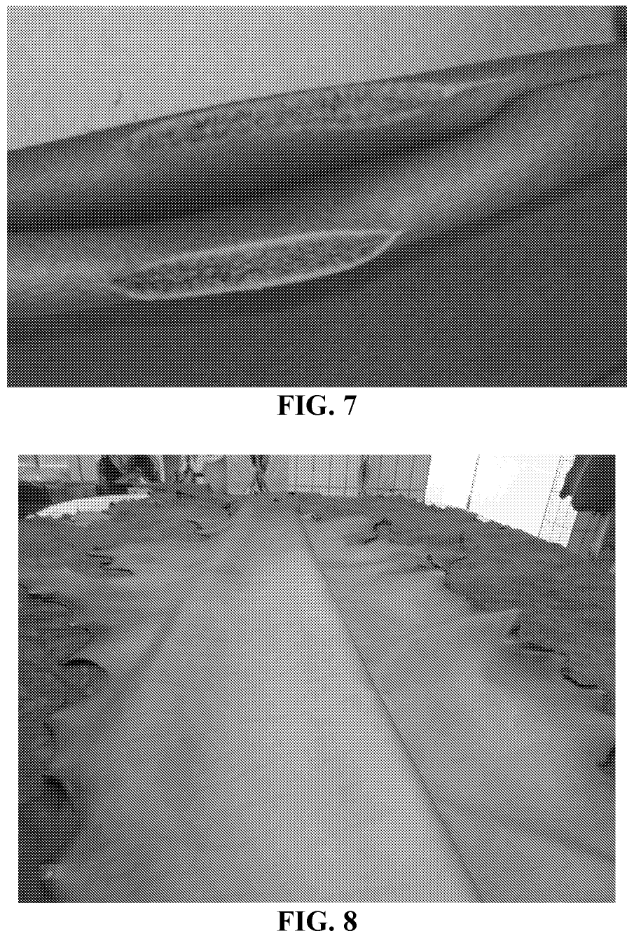

Results:
No differences in softness and waxy feel.
Color in cross section was more uniform with probiotic compounds (FIG. 7: probiotic process on top)
Color was more even through the area with probiotic compounds (FIG. 8, probiotic process on the right)
Standard was more patchy and reddish, especially in the flanks.

### Example 5

### Soaking Bovine Salted Hides after Long Preservation

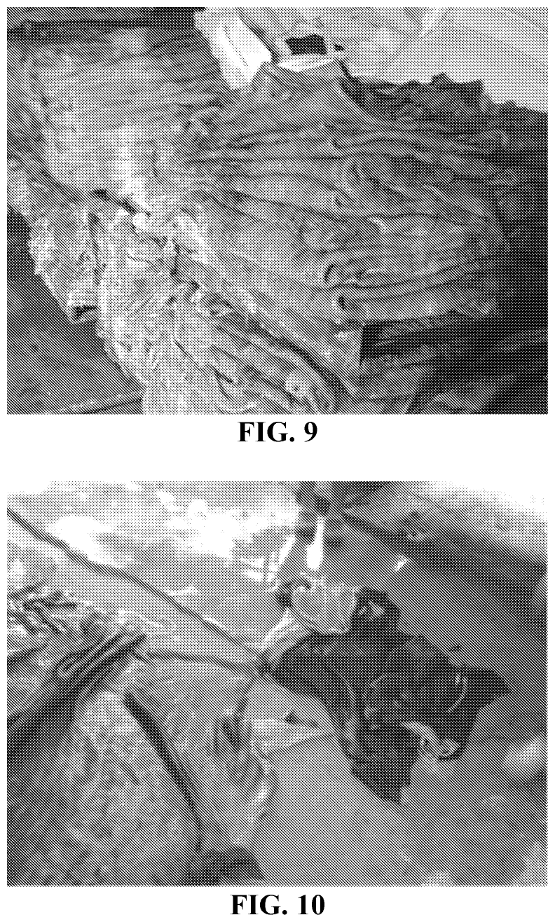

Material: In a tannery we processed 2 salted bovine hides, 60 KG. Raw material was very fatty, covered by red heat bacteria (FIG. 9) and bad smell of putrefaction.

Process:

Pre-soaking: 0.3% Probiotic compounds +0.4% after one hour and overnight in drum, replacing 2% NaCl, 0.2% enzymatic soaking agent, 0.2% soda ash and 0.15% degreaser agent based on a blend of anionic and non-ionic surfactants. Unpleasant smell disappeared.

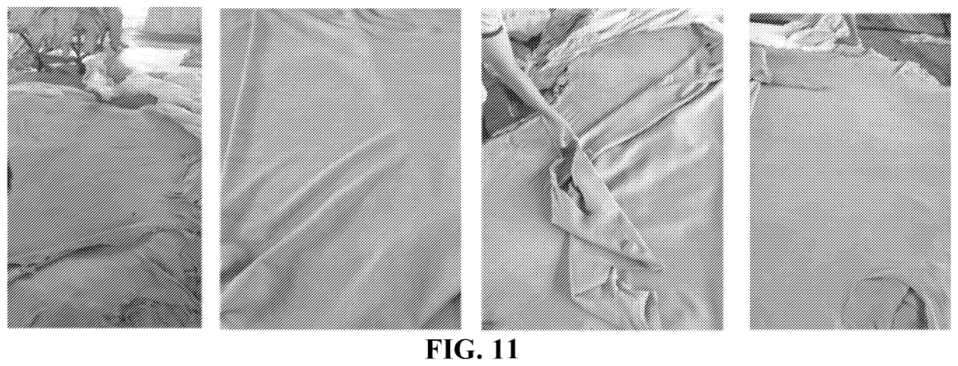

Soaking: 0.4% Probiotic compound, replacing 0.1% bactericide, 0.3% soaking agent (anionic surfactant), 0.05% enzymatic soaking agent (protease) and 0.2% degreaser agent based on a blend of anionic and non-ionic surfactants. After 12 hours soaking (FIG. 10), hides were added to a drum of tannery production. Wet blue was evaluated with comparable results as standard production (FIG. 11)

### Example 6

### Degreasing Entrefino Sheep Skins without Pickling

Material: 2×50 skins of Entrefino sheep skins (50 KG delimed weight), de-wooled and de-limed.

Degreasing: 5% % Probiotic compound and 4% emulsifier (surfactant of low HLB based on a fatty alcohol ethoxylated).

2 washes: with 0.2% emulsifier.

Bating: traditional enzymatic bating agent and 0.3% emulsifier.

Tanning: 0.1% probiotic compound previous to the tanning agent.

50 skins were processed for wet-blue and 50 skins for wet-white (organic tanning).

In both cases, 2 pickling and 1 depickling were not necessary, avoiding between 10% and 15% of NaCl, strong acids like sulfuric or formic acid and soda ash.

Analytical tests (ISO 4048) showed ˜1% matter soluble in dichloromethane (within the specifications).

### Example 7

### Short Preservation of Fresh Bovine Hides without Bactericides

Material: 23 pieces of fresh bovine hides (European breed).

Process: After flying (skin pulling, stripping) in the slaughterhouse, hides were trimmed and sent to the tannery and fleshed immediately at the arrival. Drums was uploaded with 700 KG of fresh and green fleshed hides and washed with 150% (on fleshed weight) with water at 23° C. during 10 minutes at 2 rpm. Drained and was again with 100% water at 23° C. and 0.05% of Probiotic biochemical to was blood, during 10 minutes at 2 rpm. Drained. New 100% water float at 23° C. and 0.3% Probiotic biochemical, running 10 minutes at 1 rpm and overnight (about 10 hours) running 1 minutes every hour (1 rpm). Temperature did not change overnight. Antiseptics (bactericides) were not applied. Typical bactericides are based on potassium or sodium dimethildithiocarbamate.

Result: Preservation of hides were evaluated in the morning and damages were not observed: no hair slip and no putrefaction odors were perceived.

### Example 8

### Better Up-Taking of Chromium in Tanning Wet-Blue

Material: 225 pieces of fresh bovine hides (European breed).

Process: Presoaking with 0.3% Probiotic biochemicals. Main soaking 0.4% Probiotic biochemicals. Unhairing/liming following tannery standards. Hides were unloaded and limed fleshed and combined with another production drum for deliming and chrome tanning.

Wet blue was fuller in flanks with no drawn marks and neck wrinkles were less marked compared with fully standard process.

Samples were taken from necks and bulls to determine chromium oxide content according to ISO 5398-1 method. Standard averages: 4.9% and 4.65% probiotic process and standard process respectively. Five percent better up-taking soaking with probiotic biochemicals. Crust leather with probiotic biochemicals resulted in fuller fill and color was more uniform and bright.

### Example 9

### MALDI-TOF Analysis

The fermentation products of the invention were analyzed for their fingerprint characterization using a Voyager DE STR MALDI-TOF MS system. Three different embodiments of the fermentation products were analyzed: as a wetting, dispersing, or solarizing agent. Five analyses were conducted in positive mode over the following ranges; 50-1,000, 500-5,000, 3,000-12,000, 10,000-80,000 and 10,000-150,000 m/z. The compositions were screened in negative mode for each mass range. Post-acquisition processing was done using the software, Data Explorer Version 4.0. Commercial mass standard solutions or bovine serum albumin were used to calibrate the sample spectra. Mass standards were analyzed for each m/z range and were internally calibrated. The sample spectra were externally calibrated with adjacent mass standards. The results are shown in FIGS. 12-14.

The examples given above are representative and those conversant in the art will realize that it should not restrict the scope of the invention.

1. A probiotic biochemical composition comprising a microorganism fermentation broth with a defined fingerprinting profile from metagenomics and metabolomics analysis, said probiotic biochemical composition comprising:
Total Lactic Acid Bacteria counts of between 1.0E+4 to 1.0E+6 CFU/mL;
Purple sulfur bacteria; and
being essentially free of pathogenic or putrefactive microorganisms,
wherein said fermentation broth is obtained by fermentation of probiotic microorganisms in a medium under defined fermentation conditions;
wherein said probiotic biochemical composition has a pH value of <4.0 with activity in pH range from 2 to 12, percent total acidity between about 0.6 and about 3.2, and an Emulsification Index value between about 40% and about 75%.

2. The composition of claim 1, said fermentation broth further comprising at least one of acetic acid, lactic acid, fatty acid, and/or small chain peptides.

3. The composition of claim 1 or 2, comprising at least three microorganisms, wherein the microorganisms are co-cultured, and wherein at least one microorganism is selected from the group consisting of purple non-sulfur bacteria, chromatianeae, green sulfur bacteria, colorless sulfur bacteria, filamentous green bacteria, and combinations thereof, and wherein at least two microorganisms are selected from the group consisting of Bacillus species, Bifidobacterium species, Bifidobacterium species, Enterococcus species, Lactobacillus species, Rhodopseudomonas species, Saccharomyces species, and combinations thereof.

4. The composition of any one of claims 1-3, wherein the probiotic biochemical composition has an HLB value of from about 7 to about 10.

5. The composition of any one of claims 1-3, wherein the probiotic biochemical composition has an HLB value of from about 13 to about 18.

6. A method of producing leather comprising contacting an animal hide with a probiotic biochemical composition according to any one of claims 1-5.

7. The method of claim 6, comprising contacting said hide with said composition during at least one stage of leather tanning selected from the group consisting of raw hide preservation, pre-soaking, soaking, unhairing, de-liming, bating, degreasing, tanning, wetting back, retanning, and/or fatliquoring.

8. The method of claim 7, wherein said probiotic biochemical composition is used for replacing and/or reducing at least one chemical biocide, enzyme, surfactant, sodium sulfide, trivalent chromium, and/or fatliquoring agent used during at least one of said stages.

9. The method of claim 7, wherein said probiotic biochemical composition is used for improving performance of at least one chemical biocide, enzyme, surfactant, sodium sulfide, trivalent chromium, and/or fatliquoring agent used during at least one of said stages.

10. The method of any one of claims claim 7-9, wherein an amount of sodium sulfide used in unhairing is reduced by at least 30%.

11. The method of any one of claims claim 7-10, wherein synthetic fatliquor and/or petroleum based chemical surfactants is reduced by at least 10%, thereby reducing a carbon footprint of said process.

12. The method of any one of claims claim 7-11, wherein exhaustion of an amount of trivalent chromium used in said tanning stage is improved, thereby reducing or eliminating hexavalent chromium contaminants from an effluent during said tanning stage.

13. The method of any one of claims claim 7-12, wherein said probiotic biochemical composition is not a biocidal, and wherein high microbial loads are observed during said soaking stage without hide damage.

14. The method of any one of claims 6-13, wherein said probiotic biochemical composition is effective at a ratio of between 0.1 parts to 6 parts by weight dissolved in 100 litres of water during said contacting.

15. The method of any one of claims 6-14, wherein said probiotic biochemical composition further comprises at least one of a deodorizer extracted from essential oils, and/or a stabilizing agent.

16. The method of any one of claims 6-15, wherein said probiotic biochemical composition increases solubility of sparingly-soluble molecules in water.

17. A method of reducing sodium sulfide in the production of leather products, said method comprising unhairing an animal hide by contacting the hide with lime, a probiotic biochemical composition, and an amount of sodium sulfide for an effective period of time to release hair from said hide, wherein the amount of sodium sulfide used in said contacting, is reduced by at least 30%, said probiotic biochemical composition comprising:
a microorganism fermentation broth with a defined fingerprinting profile from metagenomics and metabolomics analysis;
Total Lactic Acid Bacteria counts of between 1.0E+4 to 1.0E+6 CFU/mL;
Purple sulfur bacteria; and
being essentially free of pathogenic or putrefactive microorganisms,
wherein said fermentation broth is obtained by fermentation of probiotic microorganisms in a medium under defined fermentation conditions;
wherein said probiotic biochemical composition has a pH value of <4.0 with activity in pH range from 2 to 12, percent total acidity between about 0.6 and about 3.2, and an Emulsification Index value between about 40% and about 75%.

18. Use of a probiotic biochemical composition in the production of leather products, said probiotic biochemical composition comprising:
a microorganism fermentation broth with a defined fingerprinting profile from metagenomics and metabolomics analysis;
Total Lactic Acid Bacteria counts of between 1.0E+4 to 1.0E+6 CFU/mL;
Purple sulfur bacteria; and
being essentially free of pathogenic or putrefactive microorganisms,
wherein said fermentation broth is obtained by fermentation of probiotic microorganisms in a medium under defined fermentation conditions;
wherein said probiotic biochemical composition has a pH value of <4.0 with activity in pH range from 2 to 12, percent total acidity between about 0.6 and about 3.2, and an Emulsification Index value between about 40% and about 75%.
